(19) United States
(12) Patent Application Publication
(10) Pub. No.: US 2025/0182296 A1
(43) Pub. Date: Jun. 5, 2025

(54) METHOD FOR AUTOMATED SEGMENTATION OF AN ARTERIOVENOUS MALFORMATION

(71) Applicant: Siemens Healthineers AG, Forchheim (DE)

(72) Inventor: Annette Birkhold, Stuttgart (DE)

(21) Appl. No.: 18/964,439

(22) Filed: Nov. 30, 2024

(30) Foreign Application Priority Data
Nov. 30, 2023 (DE) 10 2023 211 997.8

(51) Int. Cl. G06T 7/162; G06T 7/00; G06T 11/20; G16H 30/40

(52) U.S. Cl. CPC G06T 7/162; G06T 7/0012; G06T 11/206; G16H 30/40; G06T 2200/04; G06T 2207/20072; G06T 2207/30101; G06T 2210/41

(57) ABSTRACT

A method of processing image data for automated segmentation of an arteriovenous malformation includes obtaining a graph representation, combining nodes of the graph representation, determining a node or combined node with a highest number of direct connections to the node or combined node as the origin node, and combining nodes connected directly to the origin node. The method includes combining, with the origin node, a branching node connected to the origin node if it fulfills: the branching node is connected directly to precisely three nodes, and the branching node is connected directly or indirectly to a further branching node that is connected directly to at least four nodes. The indirect connection includes one or more nodes that are each connected directly to precisely two nodes. The method includes carrying out the method again, beginning with the combining of the nodes, if nodes are combined with the origin node.

This application claims the benefit of German Patent Application No. DE 10 2023 211 997.8, filed on Nov. 30, 2023, which is hereby incorporated by reference in its entirety.

### BACKGROUND

The present embodiments relate to a computer-implemented method for automated segmentation of an arteriovenous malformation and also to an apparatus configured for carrying out the method, to a corresponding computer program product, and to a corresponding computer-readable memory medium.

An arteriovenous malformation (AVM) involves a malformation of the blood vessels, in which arteries are connected directly to veins. An AVM thus involves a vessel malformation that consists of a tangle of connections between the arteries taking blood to the heart and the veins draining the blood away from the heart. An exact delimitation of arteries and veins in the area of an AVM is often not possible. An exact delimitation of an AVM from a surrounding, non-malformed vessel system is likewise often not possible. Due to the optical structure of the malformation, an AVM often appears in medical images as a bundle of vessels of arterial and venous blood vessels. The center of the bundle of vessels or AVM is also referred to in medicine as a nidus. To simplify the description hereafter, the abbreviated terms AVM and AVM nidus will be used.

Medical images, which include a two-dimensional image or a three-dimensional image, for example, are based on a medical image dataset. A four-dimensional image dataset may be comprised in which there are three spatial dimensions and one dimension similar to a temporal dimension. The medical image dataset may include a plurality of individual medical images.

A medical image dataset consists of image data (e.g., in the form of a two-or three-dimensional array of pixels or voxels). Such arrays of pixels or voxels may represent color, intensity, absorption, or other parameters as a function of the two-or three-dimensional position. Such arrays of pixels or voxels may be obtained, for example, by suitable processing of measurement signals of a medical imaging modality or image scanning facility.

A medical image dataset may include a plurality of images or image slices. The slices each show a cross-sectional view of the image volume. The slices may include a two-dimensional array of pixels or voxels as image data. The arrangement of the slices in the medical image dataset may be determined by the imaging modality or by any postprocessing of the image dataset used. Slices may be artificially defined in the image volume spanned by the medical image dataset. Optionally, this may be undertaken as a function of the image data contained in the medical image dataset in order to prepare the medical image dataset in the optimal way for the subsequent diagnostic workflow.

The medical image dataset may be a radiological image dataset that shows a part of a patient's body. Accordingly, the medical image dataset may contain two or three-dimensional image data of the part of the patient's body. The medical image may be representative of an image volume or of a cross-section through the image volume. The part of the patient's body may be contained in the image volume. The part of the patient's body shown will generally include a plurality of anatomies and/or organs. If an image of a chest is taken as an example, the medical image may show lung tissue, the ribcage, lymph nodes, and others.

The medical image dataset may be an angiographic image dataset. Angiographic image data is image data that represents a blood vessel system of a patient or of a part of a patient's body. An angiographic image dataset may contain two-or three-or four-dimensional image data of the blood vessel system.

The medical image dataset may be stored in a standard image format such as the Digital Imaging and Communications in Medicine (DICOM) format and be stored in a memory or computer storage system such as a Picture Archiving and Communication System (PACS), a Radiology Information System (RIS), and the like. Whenever DICOM is mentioned in this document, it should be understood that this relates to the “Digital Imaging and Communications in Medicine” (DICOM) standard, for example, in accordance with the DICOM PS3.1 2020c standard (or a later or earlier version of this standard).

A medical imaging modality corresponds to a system that is used for creation or production of medical image data. For example, an imaging modality may be a computed tomography system (CT system), a magnetic resonance tomography system (MR system), an angiography X-ray system, a C-arm X-ray system, a positron emission tomography system (PET system), or the like.

Computed tomography, for example, is a widely used imaging method and makes use of X-rays created and captured by an instrument that rotates spatially about the object of the imaging or about the patient. This may involve what is known as cone beam computed tomography (CBCT for short), which is based on images of a cone-shaped X-ray beam, as is created, for example, by a C-arm X-ray system, or may involve fan beam computed tomography, which is based on images of a fan-shaped X-ray beam, as is created, for example, by a computed tomography system. The resulting absorption data (also referred to as raw data) is processed by computer-assisted analysis software that reconstructs more detailed images of the inner structure of the parts of the patient's body. The image datasets created are referred to as CT scans, which may represent a number of series of sequential images in order to show the inner anatomical structures in cross-sections perpendicular to the axis of the human body.

To give another example, magnetic resonance tomography (MRT) is an advanced medical imaging technique that uses the effect of the magnetic field on the movements of protons. In MRT devices, the detectors are antennas, and the signals are analyzed by a computer that creates more detailed images of the inner structures in each section of the human body.

A computer basically involves a system comprising a memory, a working memory, one or more microprocessors, and one or more input/output data interfaces.

Memory may be understood as a unit that is capable of storing data and information. This may be any type of memory medium, including, but not restricted to, hard disks, solid-state drives, optical drives, magnetic drives, flash memory, and other forms of non-volatile or volatile memory.

Working memory, also known as random access memory (RAM), may be understood as a temporary memory unit that is capable of temporarily storing program elements, data, and information to which there is access during the execution of a program or process. This may be any type of RAM including, but not restricted to, DRAM, SRAM, SDRAM, and other forms of volatile memory.

A microprocessor may be understood as a unit that is capable of executing commands and managing processes. This may include any type of processor, including, but not restricted to, single-core processors, multi-core processors, graphics processors, digital signal processors, and other forms of processors or microcontrollers.

An input/output data interface (or also I/O data interface) may be understood as a unit that is capable of transmitting data and information between various components or systems. This may include any type of data transmission interface, including, but not restricted to, USB, HDMI, VGA, DVI, Ethernet, Wi-Fi, Bluetooth, and other forms of wired or wireless data transmission interfaces.

A graph is a term from graph theory. A graph is understood as an abstract structure that represents a set of objects along with the connections existing between the objects. The mathematical abstractions of the objects are referred to in such cases as nodes. The pairs of connections between objects are referred to as edges. An edge in this sense is a direct connection between two nodes. In the description below, instead of the term edge, the synonymous term direct connection between nodes is therefore used. A graph may be represented graphically, for example, in that the nodes are shown by points and the edges by lines.

Segmentation is understood in medical imaging as a process in which specific structures or regions within images are identified or established. Segmentation may serve, for example, to determine image regions that represent specific anatomical features, such as, for example, vessels or bones or internal organs. A segmentation may be helpful, for example, in an examination of a vessel in angiography in order to trace the course of the blood vessels or to measure the diameter of the vessels.

The segmentation of an arteriovenous malformation (AVM) including the AVM nidus is of considerable importance for the diagnosis and treatment of the AVM. The normal blood flow may be interrupted by the AVM nidus, for example, and thus, the perfusion of tissue and the supply of oxygen is prevented. The segmentation of the AVM nidus in medical image data represents a challenge since AVMs have complex and irregular morphologies. Added to this, the appearance of AVMs varies from patient to patient.

The accurate segmentation of an AVM nidus is of decisive importance for treatment planning. It helps in this case to determine size and location and to identify feeding arteries as well as draining veins. With aid of this information, the treatment strategy that is best suited is determined (e.g., surgery, radio surgery, or endovascular embolization).

AVM nidus segmentation may be employed, for example, for: planning the region (AVM nidus region) to be treated surgically or radio surgically planning endovascular embolization, in which AVM nidus, feeding blood vessels, and draining blood vessels are to be determined in detail 3D overlay while carrying out an embolization in order to determine whether the entire AVM nidus has been embolized (and the risk of a rupture associated therewith)

A particular difficulty in the segmentation of AVM nidi is caused by the high variability of their size, shape, and appearance. This variability and the comparatively low number of cases (e.g., not more than 50 cases per clinic and year) make the application of machine learning algorithms more difficult.

A method for automatic segmentation of an AVM is known from the publication Babin et al. 2018 Skeletonization method for vessel delineation of arteriovenous malformation. Computers in Biology and Medicine 93 (2018) 93-105. Babin et al. initially create a graph that represents a blood vessel system from a medical image dataset. The nodes of the graph are analyzed with respect to the number of their respective edges. The number of edges of a node is also referred to in this case as a grade (e.g., a node with five edges is referred to as a fifth grade node). The node or the node combined from a plurality of nodes with the most edges (e.g., the highest grade node) is considered as the AVM nidus. A region of the graph with 5 or more edges and connection to the node with the most edges is considered as the nidus region.

Further, there is work being undertaken currently on methods for automatic segmentation using deep learning algorithms.

The accuracy of the segmentation of the AVM nidus region is not sufficient, however, for diagnostic and therapeutic purposes.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a computer-implemented automatic segmentation of an arteriovenous malformation (AVM) along with AVM nidus in a 3D image dataset that has an improved accuracy is provided. As another example, such an automatic segmentation that is able to be carried out efficiently and quickly with a computer is also provided.

The computer-implemented method of image processing for automated segmentation of an arteriovenous malformation of the present embodiments includes the following acts: obtaining a graph representation of a vessel system including a node and connections, where obtaining may be understood as determining with the aid of an image dataset or receiving from a data memory or a cloud or receiving from a facility for processing image data; S5) combining all nodes of the graph representation connected directly to one another that are connected directly to two or more nodes, into one combined node; S6) determining the highest number of direct connections to other nodes that have nodes or combined nodes of the graph representation, and determining a node or combined node with the highest number of nodes connected directly to it as the origin node, where the highest number is to be understood as the maximum number of direct connections that occur for nodes of the graph representation; S7) and combining all nodes connected directly to the origin node that fulfill one of the following conditions, with the origin node-the nodes are connected directly to precisely two nodes, or the nodes are connected directly to at least four nodes.

In accordance with the present embodiments, the method includes the subsequent further acts: S8) combining a branching node connected to the origin node with the origin node if it fulfills the two conditions below-the branching node is connected directly to precisely three nodes, and the branching node is connected directly or indirectly to a further branching node that is connected directly to at least four nodes, where the indirect connection includes one or more nodes that are each connected directly to precisely two nodes; and carrying out the method once again, beginning with act S7), if in act S8), nodes are combined with the origin node.

Subsequently, the graph representation established in act S8 is provided for display by a display device and/or for execution of further image processing acts.

By the combination of branching nodes with the origin node in act S8, by comparison with the prior art, additional further nodes are combined with the origin node, the AVM nidus. It is an insight of the present embodiments that the condition that is tested in this act of the method takes account of the anatomical peculiarities of blood vessel systems with arteriovenous malformation in a better adapted way. The combination of nodes with the AVM nidus in accordance with this act of the method therefore brings about a better match between the segmentation and the actual AVM nidus, which takes better account of the anatomical peculiarities than is the case in methods known in the prior art.

Further, the term combine may be, in this case, an assignment of the nodes to one another. The assignment to one another may be undertaken in the graph by all nodes being replaced by a single node that represents the nodes assigned to one another. The assignment to one another may also be undertaken by the nodes assigned to one another being interpreted as a uniform region of which the connections to further nodes is handled as if the region represented a single node. In each case, connections that exist between the nodes of a combined node or between a combined node and further nodes are handled as if the combined node were an individual node. For the sake of simpler description, combined nodes are represented below by a single node.

In a development of the method, in act S9 following act S8, the nodes of an end component connected to the origin node are combined with the origin node. An end component in this case includes an end node that is connected directly to precisely one further node. An end component may also include a node or further nodes that are connected directly to precisely two further nodes.

Local groups of nodes and edges within a restricted section of the graph representation are referred to as components. Components are characterized by the nodes and edges from which they are formed having a common property connecting them.

Through the combination of non-connected end components with the origin node, by comparison with the prior art, additional further nodes are added to the AVM nidus in the graph. End components may come into being, for example, when further connections are not able to be recognized or not able to be recognized without errors or able to be recognized only with low contrast in the 3D image dataset underlying the graph. End components may accordingly arise, for example, when a vessel is very small and, on account of this, cannot be resolved and shown in the image, or when the image data has too little contrast, or when a stenosis narrows a vessel, or when a vessel actually ends.

It is an insight of the present embodiments that the condition that is tested in this act of the method takes account of the anatomical peculiarities of blood vessel systems with arteriovenous malformation in a better adapted way. The combination of end components with the AVM nidus in accordance with this act of the method therefore brings about a better match between the segmentation and the actual AVM nidus, which takes better account of the anatomical circumstances than is the case in methods known in the prior art.

In a development of the method, in act S10 following act S8, nodes of a large component connected to the origin node are combined with the origin node. A connected large component consists of a plurality of nodes connected to one another. In this case, the number of nodes within the large component that are connected directly to precisely three nodes are to be less than the number of nidus connections of the large component to the origin node plus two, and the nodes of the large component may exclusively be connected directly to the origin node and to further nodes of the large component.

Through the condition for large components, which are tested in this act S10 of the method, local clusters are then combined with the AVM nidus when, in accordance with the condition, in relation to their nodes directly connected three times, they have sufficiently many connections to the AVM nidus. If sufficiently many conditions to the AVM nidus exist, such components are thus not attributed any independent significance separate from the AVM nidus.

It is an insight of the present embodiments that the condition that is tested in this act of the method takes account of the anatomical peculiarities of blood vessel systems with arteriovenous malformation in a better adapted way. The combination of connected large components with the AVM nidus in accordance with the present embodiments therefore brings about a better match between the segmentation and the actual AVM nidus, which takes better account of the anatomical circumstances than is the case in methods known in the prior art.

An advantageous development of the method concerns the case in which a plurality of nodes or combined nodes has the highest number of nodes connected directly to it. In this case, in act S6, a number of this plurality of nodes or combined nodes are selected as the origin node. Then, for each of the selected origin nodes, one or more of the subsequent method acts are carried out separately in each case.

If a number of nodes or combined nodes with the highest number of directly connected nodes are established, it is not able to be clearly determined beforehand which of the nodes is the origin node (e.g., which of the nodes corresponds to the AVM nidus). By separately carrying out the method for each or at least a number of the origin nodes in question, a separate segmentation is carried out automatically for each of the selected origin nodes. The further method acts may then either produce a common AVM nidus graph, or a number of different segmentations are obtained and then output for the user of the method.

In a development of the method, depending on the result of the method acts carried out beforehand, one or more nodes or combined nodes with a number of directly connected nodes that is less than the highest number previously established are selected as origin nodes. Subsequently, one or more of the method acts, beginning with act S7, is carried out separately in each case for each of these selected origin nodes.

In accordance with this development of the method, then, for example, when the node or nodes or combined nodes with the highest number of nodes connected to them do not lie in the AVM nidus as a result of the segmentation method, the method is repeated with nodes or combined nodes with fewer direct connections. The result of the method acts carried out previously, which gives rise to this new selection of origin nodes, is accordingly that the origin node or nodes selected previously did not lie in the AVM nidus. Accordingly, incorrect results of the method acts carried out may also give rise in another way to this new selection of origin nodes. For example, a user of the method, depending on the result, may make a user input, as a result of which, the new selection of origin nodes is made. When the selection of origin nodes according to the original criterion of the highest grade leads to an error-prone result of the method, a new selection of origin nodes of lower grade is made, and the further method is run through once again.

In a development of the method, the graph representation including the nodes and connections is established by the acts preceding the act S5: S1) receiving a 3D image dataset; S2) establishing a representation of a vessel system in the 3D image dataset; S3) establishing a line representation of the vessel system; and S4) establishing a node and connections between the graph representation of the line representation comprising nodes. The 3D image dataset may involve any given type of angiographic image dataset.

The 3D image dataset may be created, for example, by a computed tomography system or by a C-arm X-ray system. Fan beam computed tomography or cone beam computed tomography may be involved, for example.

The representation of the vessel system may be established, for example, by digital subtraction angiography (DSA). In DSA, image data in which blood vessels do not contain any contrast medium is subtracted from image data in which blood vessels contain contrast medium. The result of the subtraction is image data that exclusively represents blood vessels. The representation of the vessel system may also be established by another type of image data processing (e.g., by segmentation of an angiographic magnetic resonance tomography). Numerous methods are known for representation of the vessel system.

Establishing a line representation of the vessel system may also be referred to as skeletonization. In this case, the planar representation of a blood vessel in 2D image data or the spatially expanded representation of a blood vessel in 3D image data is converted into a line representation. The line representation thus represents the course of a blood vessel, but not necessarily its spatial extent or its diameter. What is important about this is fundamentally merely that the lines that are selected lie within the planar or spatial representation of a blood vessel. Frequently, lines in the middle of the planar or spatial representation of the blood vessel are selected. Such a representation is referred to as a centerline representation. Numerous methods are known for establishing a line representation of a vessel system.

A graph representation including connections and nodes is essentially established by crossing points in the line representation of the vessel system being represented by nodes and lines by connections, where the connections may be shown as straight lines. Numerous methods are known for establishing a graph representation.

In a development of the method, in a subsequent act S11, those voxels of the 3D image dataset are determined that correspond to the nodes and origin nodes established in the graph representation. To do this, for each node or origin node established, the point that lies next in the line representation of the vessel system is determined.

The assignment of the AVM and AVM nidus segmented in the graph representation to the original 3D image dataset is required so that the segmentation is spatially comprehensible for the observer and is able to be assigned spatially to the vessel system shown in the 3D image dataset. In this way, the segmentation is made usable for diagnosis and therapy and comprehensible for spatial orientation and navigation. Since a graph representation basically shows a logical representation of the vessel system, the graph representation does not necessarily reproduce a spatially correct assignment of nodes and connections. The graph representation is based, however, on the line representation of the vessel system, since it has been established from the latter. Thus, an assignment of nodes and connections of the graph representation to crossing points and lines of the line representation is produced right from the start. Making use of the existing assignment in order to assign segmented features of the graph representation to the line representation takes little effort and avoids assignment errors.

Then, in one or more following acts, segmented features of the line representation of the vessel system are assigned to the original representation of the vessel system or to the original 3D image dataset. Since here too the respective assignment is given right from the start, nodes and connections may also be assigned here with little effort and while avoiding assignment errors. For example, segmented features of the graph representation may initially be assigned to the line representation and only thereafter to an original representation of the vessel system with less effort and fewer errors than a direct assignment of features of the graph representation to the original representation of the vessel system or to the original 3D image dataset.

A further development of the present embodiments consists of a facility for processing image data including a computer unit that is configured to carry out a method of the present embodiments. The computer unit may involve a microprocessor, a multi-core microprocessor (multi-core CPU), a distributed processor arrangement, a correspondingly configured graphics processor unit, a network resource (e.g., cloud computing), or similar conventional facilities.

A further development of the present embodiments consists of an imaging facility including an imaging modality as well as a facility for processing image data configured as explained above, where the imaging modality is configured to provide the image data. The imaging modality may involve a computed tomography system, a C-arm X-ray system, another X-ray system, a magnetic resonance tomography system, or another conventional imaging modality for creating angiographic image data.

A further development of the present embodiments consists of a computer program product including program elements that, when they are loaded into a working memory of a computer unit of a facility for processing of image data, cause the computer unit to carry out the method acts explained above. The computer program product may include program instructions in source code, in compiled code (e.g., object code), or in other conventional program languages.

A further development of the present embodiments consists of a computer-readable memory medium on which program elements are stored that are suitable to be read and executed by a computer unit, and which, when they are loaded into a working memory of a computer unit, cause the computer unit to carry out the method acts explained above. The memory medium may involve a hard disk, a diskette or CD, a solid state memory drive (SSD), a USB memory, network memory (e.g., cloud memory), or another conventional data memory.

### BRIEF DESCRIPTION OF THE DRAWINGS

FIG. 1 shows an imaging facility of the prior art;

FIG. 2 shows a graph representation of the prior art;

FIG. 3 shows a flow diagram of the prior art;

FIG. 4 shows nodes with three connected nodes;

FIG. 5 shows short open ends;

FIG. 6 shows large regions; and

FIG. 7 shows a flow diagram.

### DETAILED DESCRIPTION

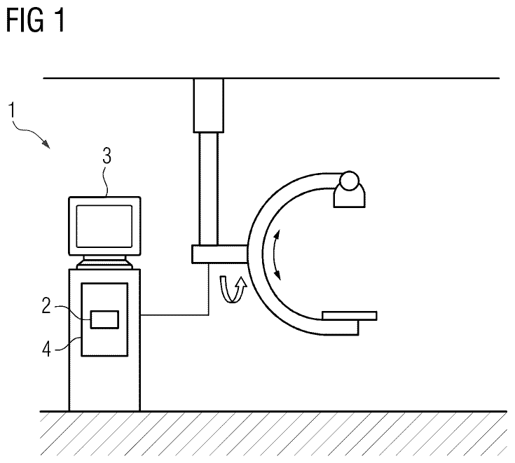

FIG. 1 shows a schematic of an imaging facility that is suitable for carrying out the present embodiments. The imaging facility includes an imaging modality. The imaging modality involves a C-arm X-ray system 1. The C-arm X-ray system 1 is merely given by way of example. It is important that the imaging modality used is suitable for creating angiography image data. Thus, for example, a computed tomography system or a magnetic resonance tomography system may also be used to carry out the present embodiments.

The imaging facility further includes a facility for processing image data 3. The facility for processing image data 3 accepts image data from the imaging modality and processes the image data. The image data is processed by the computer unit 4. The computer unit 4 is connected to a working memory 2. Program instructions may be loaded into the working memory 2, by which the computer unit 4 is caused to carry out the method acts encoded by the program instructions. Depending on the type of imaging modality, the processing of image data may include a pre-processing of image data, a three-dimensional image reconstruction, a denoising, and other post-processing steps. What is important is merely that the processing of the angiographic image data includes a representation of blood vessels (e.g., by a segmentation method or by a DSA method).

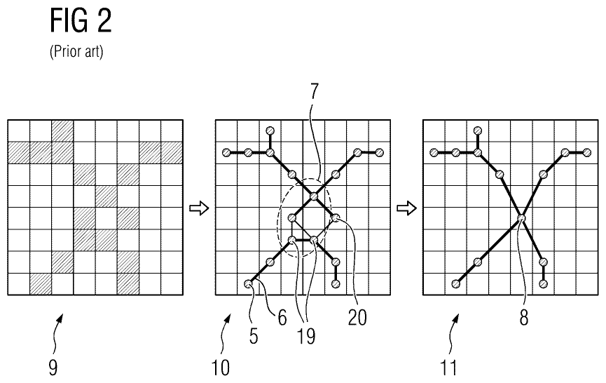

FIG. 2 shows a schematic of the processing of angiographic image data for automatic segmentation of an AVM or an AVM nidus in accordance with the prior art. The starting point is a line representation 9 of blood vessels of an AVM shown on the left in the figure. The lines are shown in a very coarse image grid in order to make the transmission into the graph representation 10 shown in the middle, which uses the same coarse image grid, easier to understand. The line representation 9 shown may be obtained by any given method known in the prior art.

The graph representation 10 shown in the middle is created from the line representation 9 shown on the left. The same coarse grid is used as in the line representation 9 shown on the left. In the graph representation 10 shown in the middle, nodes 5 are shown as points, and edges or connections 6 are shown as lines. Nodes 5 may have one or more neighbors (e.g., one or more nodes 5 connected directly to them). Visible in roughly the center of the grid is a region 7 enclosed by a dashed line with a high number of nodes 5 and connections 6. The region 7 appears for an observer from its arrangement and interconnectedness to a certain extent as the origin or starting point of the graph representation 10.

Shown on the right, as the result of an automatic segmentation of the AVM nidus, is a segmented graph representation 11. By the automatic segmentation in accordance with the prior art, initially, nodes 19 are established that are connected directly to more than two nodes 5. Such multiply directly connected nodes 19 are combined with other multiply directly connected nodes 19 to which they are connected directly. Shown enclosed by a dashed line in the center of the graph representation 10 is a region 7 in which a plurality of such multiply directly connected nodes 19 is located. These nodes 19 are combined into a single node 8. The node 20 adjoining this region merely has two direct connections 6 to further nodes 5. The node 20 directly connected twice is not combined in this step with the multiply directly connected nodes 19. The node 20 initially remains as a node directly connected twice and is only added to the AVM nidus in a later step (e.g., in the act S7 explained below). The node 8 appears for an observer through its arrangement and interconnectedness as the origin or starting point of the graph representation 10.

Basically, one or more such combined nodes may occur in a graph representation 10 of a blood vessel system. That node or combined node that has the highest number of directly connected nodes (e.g., the node or combined node with the highest grade) is defined as the origin node 8.

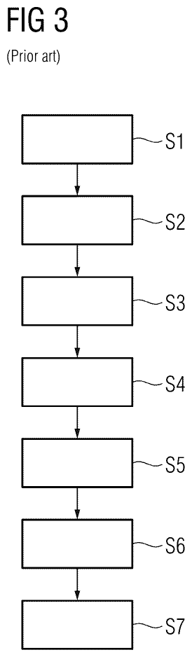

FIG. 3 shows the automatic AVM segmentation in accordance with the prior art as a schematic flow diagram. In act S1, a 3D image dataset is received from a facility for processing image data 3. In act S2 (e.g., by a segmentation method or a DSA method), a representation of a vessel system in the 3D image dataset is established. In act S3, a line representation 9 of the vessel system is established. In act S4, a graph representation 10 of the line representation 9 including nodes 5 and connections 6 is established. In act S5, multiply connected nodes 19 of the graph representation 10 are combined, as previously described, to form one or more combined nodes. In act S6, the highest number of direct connections to other nodes 5 that have nodes 5 or combined nodes 8 of the graph representation 10 is established. The node 5 or combined node 8 with the highest number of nodes 5 connected directly to the node 5 or combined node 8 is determined as the origin node 8. In act S7, all nodes 5 connected to the origin node 8 that are either connected directly to two further nodes 5 or are directly connected to more than a minimum number of nodes 5 are established. The minimum number may be higher than three and may, for example, be four.

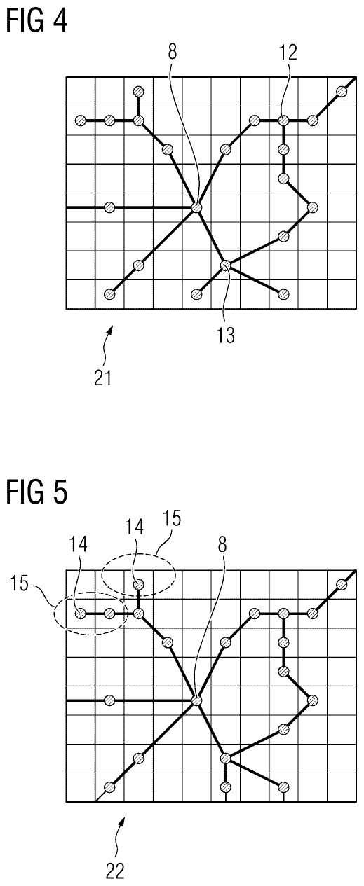

FIG. 4 schematically explains method acts of the computer-implemented method for automatic segmentation of an AVM or AVM nidus. The segmentation of the present embodiments may use as its starting point the result of a segmentation according to the prior art. It is immaterial here whether the starting point has been established by the previously described method or by another method known in the prior art. It is merely of importance that a segmented graph representation 11 of the AVM nidus with segmented origin node 8 is present.

In accordance with the present embodiments, in the segmented graph representation 21, in act S8, isolated branching nodes 12 that are defined by two conditions are identified. First, such isolated branching nodes 12 have three nodes connected directly to them. Second, the isolated branching nodes 12 have a neighboring branching node 1 that is connected directly to at least four nodes. Such a neighboring branching node 13 may be connected directly to the isolated branching node 12. Such a neighboring branching node 13 may also be connected indirectly to the isolated branching node 12 via one or more further nodes that are connected directly to precisely two nodes. In other words, the isolated branching node 12 may be linked indirectly to the branching node 13 via one or more non-branched nodes.

If, according to the criteria described above, one or more isolated branching nodes 12 are identified, these are combined with the origin node 8.

If, as described above, one or more isolated branching nodes 12 have been combined with the origin node 8, the method is subsequently carried out once again, beginning with the act S7.

FIG. 5 explains schematically further method acts of a computer-implemented method for automatic segmentation of an AVM or AVM nidus in accordance with a development of the present embodiments. After act S8 is ended, in accordance with the development, in act S9 in the graph representation 22, regions are identified as open ends 15 when the regions fulfill two conditions. First, the regions include an end node 14 that is solely connected directly to one further node. Second, above and beyond this, the regions include either no nodes or exclusively such nodes as, for their part, are merely connected directly to two nodes.

If, as described above, one or more open ends 15 are identified, the nodes 14 and, where necessary, further nodes of these open ends 15 are combined with the origin node 8.

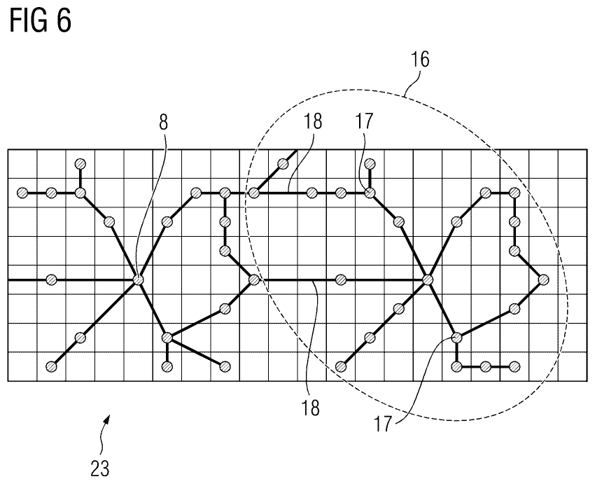

FIG. 6 explains schematically further method acts of a computer-implemented method for automatic segmentation of an AVM or AVM nidus in accordance with a development of the present embodiments. In accordance with the development, in accordance with act S10, connected regions are identified in the graph representation 23 as large components 16 if the connected regions fulfill the following two conditions. First, the number of nodes 17 of the large component 16 that are connected directly to precisely three further nodes 5 of the large component 16 is to be less than the number of the nidus connections 18 increased by two, via which nodes 5 of the large component 16 are connected to the AVM nidus. Second, the nodes 5 of the large component 16 may exclusively be connected directly to the origin node 8 or to other nodes 5 of the large component 16.

If one or more large connected regions 16, as described above, are identified, these are added to the origin node 8 and combined with the node.

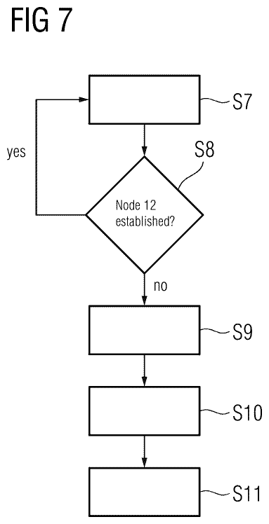

FIG. 7 shows the automatic AVM segmentation in accordance with the present embodiments as a schematic flow diagram. The method of the present embodiments uses, as its starting point, a graph representation 10 of the AVM nidus. In act S7 already described, connected nodes 5 are determined and combined with the origin node 8. In act S8, branching nodes 12 connected to the origin node 8 are sought. If such branching nodes 12 are found, the branching nodes 12 are combined with the origin node 8, and the method is run through again as from act S7.

If no such branching nodes 12 are found, the method is continued with act S9. In act S9, short open ends 15 are established and are combined where necessary with the origin node 8.

Thereafter, in act S10, large regions 16 connected to the origin node 8 are sought and combined where necessary with the origin node 8. With regard to act S10, in this case, it is merely important that it is carried out after the preceding act S8. However, act S10 does not necessarily have to be carried out after act S9. The order in which acts S10 and S9 are carried out may be selected as required.

In order to be able to show or to use the automatically established AVM nidus segmentation in the original underlying 3D image dataset, in act S11, there is an assignment of those voxels of the 3D image dataset that correspond to the nodes 5 established in the graph representation 10 and the origin node 8. In this case, the established nodes from the graph representation 10 are assigned to the line representation 9 in the sense of a mapping. Through this assignment, the nodes 5, 8 from the logical coordinate system of the graph representation 10 are mapped into the spatial coordinate system of the line representation 9 and thus assigned to the corresponding spatial coordinates. In this case, for each node 5, 8 established in the graph representation 10, the spatial point lying closest to it in the line representation 9 is determined.

In these patent application documents, independent of the grammatical term usage of a specific person-related term, individuals with male, female, or other gender identities should be included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for processing image data for automated segmentation of an arteriovenous malformation, the method being computer-implemented and comprising:
obtaining a graph representation comprising nodes and connections of a vessel system;
combining nodes of the graph representation connected directly to one another that are connected directly to more than two nodes, such that a combined node is formed;
determining a highest number of direct connections to other nodes that have nodes or combined nodes of the graph representation, and determining a node or combined node having a highest number of nodes connected directly to the node or the combined node as the origin node;
combining, with the origin node, nodes connected directly to the origin node that fulfill:
the nodes are connected directly to precisely two nodes; or
the nodes are connected directly to at least four nodes;
combining, with the origin node, a branching node connected to the origin node when the branching node fulfills:
the branching node is connected directly to precisely three nodes; and
the branching node is connected directly or indirectly to a further branching node that is connected directly to at least four nodes, wherein the indirect connection comprises one or more nodes that are each connected directly to precisely two nodes;
carrying out the method once again, beginning with the combining of the nodes connected directly to the origin node, when nodes are combined with the origin node; and
providing a graph representation established in the combining of the branching node with the origin node for display by a display device, for carrying out further image processing steps, or for a combination thereof.

2. The method of claim 1, further comprising:
combining nodes of an end component connected to the origin node with the origin node,
wherein the end component comprises an end node that is connected directly to precisely one further node, and comprises no nodes or only such nodes as are connected directly to precisely two further nodes.

3. The method of claim 1, further comprising:
combining nodes of a large component connected to the origin node with the origin node,
wherein the large component consists of nodes connected to one another,
wherein a number of nodes within the large component that are connected directly to precisely three nodes is less than a number of nidus connections of the large component to the origin node plus two, and
wherein the nodes of the large component are directly connected exclusively to the origin node and to other nodes of the large component.

4. The method of claim 1, wherein, when a plurality of nodes or combined nodes has the highest number of nodes connected directly, in the determining, a number of nodes or combined nodes of the plurality of nodes or combined nodes are selected as origin nodes, and one or more of the subsequent method steps are carried out, separately in each case, for each of the selected origin nodes.

5. The method of claim 1, wherein, depending on a result of the method steps carried out previously, one or more nodes or combined nodes with a number of nodes connected directly to the one or more nodes or combined nodes that is less than the highest number are selected as origin nodes, and one or more of the method steps, beginning with the combining of the nodes, is carried out, separately in each case, for each of the selected origin nodes.

6. The method of claim 1, further comprising:
receiving a three-dimensional (3D) image dataset;
establishing a representation of a vessel system in the 3D image dataset;
establishing a line representation of the vessel system based on the representation of the vessel system; and
establishing a graph representation comprising nodes and connections between the nodes of the line representation.

7. The method of claim 6, further comprising:
identifying voxels of the 3D image dataset that correspond to the nodes and origin nodes established in the graph representation,
wherein, for each node and origin node established, a point lying closest to the respective node or origin node in the line representation of the vessel system is determined.

8. A system for processing image data, the system comprising:
a computer unit configured to process image data for automated segmentation of an arteriovenous malformation, the computer unit being configured to process image data comprising the computer unit being configured to:
obtain a graph representation comprising nodes and connections of a vessel system;
combine nodes of the graph representation connected directly to one another that are connected directly to more than two nodes, such that a combined node is formed;
determine a highest number of direct connections to other nodes that have nodes or combined nodes of the graph representation, and determine a node or combined node having a highest number of nodes connected directly to the node or the combined node as the origin node;
combine, with the origin node, nodes connected directly to the origin node that fulfill:
the nodes are connected directly to precisely two nodes; or
the nodes are connected directly to at least four nodes;
combine, with the origin node, a branching node connected to the origin node when the branching node fulfills:
the branching node is connected directly to precisely three nodes; and
the branching node is connected directly or indirectly to a further branching node that is connected directly to at least four nodes, wherein the indirect connection comprises one or more nodes that are each connected directly to precisely two nodes;
carry out the process of the image data once again, beginning with the combination of the nodes connected directly to the origin node, when nodes are combined with the origin node; and
provide a graph representation established in the combination of the branching node with the origin node for display by a display device, for carrying out further image processing steps, or for a combination thereof.

9. An imaging facility comprising:
an imaging modality; and
a system for processing image data, the system comprising:
a computer unit configured to process image data for automated segmentation of an arteriovenous malformation, the computer unit being configured to process image data comprising the computer unit being configured to:
obtain a graph representation comprising nodes and connections of a vessel system;
combine nodes of the graph representation connected directly to one another that are connected directly to more than two nodes, such that a combined node is formed;
determine a highest number of direct connections to other nodes that have nodes or combined nodes of the graph representation, and determine a node or combined node having a highest number of nodes connected directly to the node or the combined node as the origin node;
combine, with the origin node, nodes connected directly to the origin node that fulfill:
the nodes are connected directly to precisely two nodes; or
the nodes are connected directly to at least four nodes;
combine, with the origin node, a branching node connected to the origin node when the branching node fulfills:
the branching node is connected directly to precisely three nodes; and
the branching node is connected directly or indirectly to a further branching node that is connected directly to at least four nodes, wherein the indirect connection comprises one or more nodes that are each connected directly to precisely two nodes;
carry out the process of the image data once again, beginning with the combination of the nodes connected directly to the origin node, when nodes are combined with the origin node; and
provide a graph representation established in the combination of the branching node with the origin node for display by a display device, for carrying out further image processing steps, or for a combination thereof,
wherein the imaging modality is configured to provide the image data.

10. A non-transitory computer-readable storage medium that stores instructions executable by one or more processors to process image data for automated segmentation of an arteriovenous malformation, the instructions comprising:
obtaining a graph representation comprising nodes and connections of a vessel system;
combining nodes of the graph representation connected directly to one another that are connected directly to more than two nodes, such that a combined node is formed;
determining a highest number of direct connections to other nodes that have nodes or combined nodes of the graph representation, and determining a node or combined node having a highest number of nodes connected directly to the node or the combined node as the origin node;
combining, with the origin node, nodes connected directly to the origin node that fulfill:
the nodes are connected directly to precisely two nodes; or
the nodes are connected directly to at least four nodes;
combining, with the origin node, a branching node connected to the origin node when the branching node fulfills:
the branching node is connected directly to precisely three nodes; and
the branching node is connected directly or indirectly to a further branching node that is connected directly to at least four nodes, wherein the indirect connection comprises one or more nodes that are each connected directly to precisely two nodes;
carrying out the method once again, beginning with the combining of the nodes connected directly to the origin node, when nodes are combined with the origin node; and
providing a graph representation established in the combining of the branching node with the origin node for display by a display device, for carrying out further image processing steps, or for a combination thereof.

11. The non-transitory computer-readable storage medium of claim 10, wherein the instructions further comprise:
combining nodes of an end component connected to the origin node with the origin node,
wherein the end component comprises an end node that is connected directly to precisely one further node, and comprises no nodes or only such nodes as are connected directly to precisely two further nodes.

12. The non-transitory computer-readable storage medium of claim 10, wherein the instructions further comprise:
combining nodes of a large component connected to the origin node with the origin node,
wherein the large component consists of nodes connected to one another,
wherein a number of nodes within the large component that are connected directly to precisely three nodes is less than a number of nidus connections of the large component to the origin node plus two, and
wherein the nodes of the large component are directly connected exclusively to the origin node and to other nodes of the large component.

13. The non-transitory computer-readable storage medium of claim 10, wherein, when a plurality of nodes or combined nodes has the highest number of nodes connected directly, in the determining, a number of nodes or combined nodes of the plurality of nodes or combined nodes are selected as origin nodes, and one or more of the subsequent method steps are carried out, separately in each case, for each of the selected origin nodes.

14. The non-transitory computer-readable storage medium of claim 10, wherein, depending on a result of the method steps carried out previously, one or more nodes or combined nodes with a number of nodes connected directly to the one or more nodes or combined nodes that is less than the highest number are selected as origin nodes, and one or more of the method steps, beginning with the combining of the nodes, is carried out, separately in each case, for each of the selected origin nodes.

15. The non-transitory computer-readable storage medium of claim 10, wherein the instructions further comprise:
receiving a three-dimensional (3D) image dataset;
establishing a representation of a vessel system in the 3D image dataset;
establishing a line representation of the vessel system based on the representation of the vessel system; and
establishing a graph representation comprising nodes and connections between the nodes of the line representation.

16. The non-transitory computer-readable storage medium of claim 15, wherein the instructions further comprise:
identifying voxels of the 3D image dataset that correspond to the nodes and origin nodes established in the graph representation,
wherein, for each node and origin node established, a point lying closest to the respective node or origin node in the line representation of the vessel system is determined.
